SEQUENCE LISTING (14 sequences)

SEQ ID NO: 1 - DNA, 156 bp, Homo sapiens
atgtgggggctcaaggttctgctgctacctgtggtgagctttgctctgtaccctgaggagatactggacacccactgggagctatggaagaagacccacaggaagcaatataacaacaaggctcttaattccatggttagttccccaactaaactg

SEQ ID NO: 2 - protein, 52 aa, Homo sapiens
MWGLKVLLLPVVSFALYPEEILDTHWELWKKTHRKQYNNKALNSMVSSPTKL

SEQ ID NO: 3 - DNA, 873 bp, Homo sapiens
atgattgtggatgaaatctctcggcgtttaatttgggaaaaaaacctgaagtatatttccatccataaccttgaggcttctcttggtgtccatacatatgaactggctatgaaccacctgggggacatgaccagtgaagaggtggttcagaagatgactggactcaaagtacccctgtctcattcccgcagtaatgacaccctttatatcccagaatgggaaggtagagccccagactctgtcgactatcgaaagaaaggatatgttactcctgtcaaaaatcagggtcagtgtggttcctgttgggcttttagctctgtgggtgccctggagggccaactcaagaagaaaactggcaaactcttaaatctgagtccccagaacctagtggattgtgtgtctgagaatgatggctgtggagggggctacatgaccaatgccttccaatatgtgcagaagaaccggggtattgactctgaagatgcctacccatatgtgggacaggaagagagttgtatgtacaacccaacaggcaaggcagctaaatgcagagggtacagagagatccccgaggggaatgagaaagccctgaagagggcagtggcccgagtgggacctgtctctgtggccattgatgcaagcctgacctccttccagttttacagcaaaggtgtgtattatgatgaaagctgcaatagcgataatctgaaccatgcggttttggcagtgggatatggaatccagaagggaaacaagcactggataattaaaaacagctggggagaaaactggggaaacaaaggatatatcctcatggctcgaaataagaacaacgcctgtggcattgccaacctggccagcttccccaagatg

SEQ ID NO: 4 - protein, 291 aa, Homo sapiens
MIVDEISRRLIWEKNLKYISIHNLEASLGVHTYELAMNHLGDMTSEEVVQKMTGLKVPLSHSRSNDTLYIPEWEGRAPDSVDYRKKGYVTPVKNQGQCGSCWAFSSVGALEGQLKKKTGKLLNLSPQNLVDCVSENDGCGGGYMTNAFQYVQKNRGIDSEDAYPYVGQEESCMYNPTGKAAKCRGYREIPEGNEKALKRAVARVGPVSVAIDASLTSFQFYSKGVYYDESCNSDNLNHAVLAVGYGIQKGNKHWIIKNSWGENWGNKGYILMARNKNNACGIANLASFPKM

SEQ ID NO: 5 - DNA, 20 bp, Homo sapiens
taacaacaaggctcttaatt

SEQ ID NO: 6 - DNA, 20 bp, Homo sapiens
atgattgtggatgaaatctc

SEQ ID NO: 7 - protein, 10 aa, Homo sapiens
QYNNKALNSM

SEQ ID NO: 8 - protein, 10 aa, Homo sapiens
MIVDEISRRL

SEQ ID NO: 9 - DNA, 28 bp, Homo sapiens
acgaagccagacaacagatttccatcag

SEQ ID NO: 10 - DNA, 28 bp, Homo sapiens
tactgcgggaatgagacaggggtacttt

SEQ ID NO: 11 - DNA, 27 bp, Homo sapiens
atgtgggggctcaaggttctgctgcta

SEQ ID NO: 12 - DNA, 27 bp, Homo sapiens
ttacagtttagttggggaactaaccat

SEQ ID NO: 13 - DNA, 27 bp, Homo sapiens
atgattgtggatgaaatctctcggcgt

SEQ ID NO: 14 - DNA, 27 bp, Homo sapiens
tcacatcttggggaagctggccaggtt

(19) United States
(12) Patent Application Publication
(10) Pub. No.: US 2004/0224341 A1
(43) Pub. Date: Nov. 11, 2004

(54) Alternatively spliced isoforms of cysteine protease cathepsin K (CTSK)

(76) Inventors: Christopher D. Armour, Seattle, WA (US); John C. Castle, Seattle, WA (US); Philip W. Garrett-Engele, Seattle, WA (US); Jason M. Johnson, Seattle, WA (US); Zhengyan Kan, Bellevue, WA (US); Patrick M. Loerch, Seattle, WA (US)

Correspondence Address: R. DOUGLAS BRADLEY, Merck & Co., Inc., Patent Department RY60-30, P.O. Box 2000, Rahway, NJ, 07065-0907, US

(21) Appl. No.: 10/836,897

(22) Filed: Apr. 30, 2004

Related U.S. Application Data
(60) Provisional application No. 60/467,586, filed on May 2, 2003.

(51) Int. Cl. C12Q 1/68; C12N 9/12; C07H 21/04

(52) U.S. Cl. 435/6; 435/69.1; 435/194; 435/320.1; 435/325; 536/23.2

(57) ABSTRACT

The present invention features nucleic acids and polypeptides encoding two novel splice variant isoforms of cysteine protease cathepsin K (CTSK). The polynucleotide sequences of CTSKsv1.1 and CTSKsv1.2 are provided by SEQ ID NO 1 and SEQ ID NO 3, respectively. The amino acid sequences for CTSKsv1.1 and CTSKsv1.2 are provided by SEQ ID NO 2 and SEQ ID NO 4, respectively. The present invention also provides methods for using CTSKsv1.1 and CTSKsv1.2 polynucleotides and proteins to screen for compounds that bind to CTSKsv1.1 and CTSKsv1.2, respectively.

This application claims priority to U.S. Provisional Patent Application Ser. No. 60/467,586 filed on May 2, 2003, which is incorporated by reference herein in its entirety.

### BACKGROUND OF THE INVENTION

The references cited herein are not admitted to be prior art to the claimed invention.

The mature human skeleton undergoes continuous regeneration or remodeling through a cyclical process of resorption of old bone and deposition of new bone in its place. Resorption of old bone is carried out by osteoclasts. Osteoclasts are large multinucleated cells that attach to the bone surface and produce an acidic environment wherein the mineral component of the bone matrix is solubilized. The underlying proteins are subsequently degraded by metalloproteinases and a cysteine protease, cathepsin K. Once the old bone has been removed, new bone is deposited by osteoblasts. Osteoblasts secrete proteins that constitute the bone matrix, mainly type I collagen, and regulate the mineralization process by controlling the deposition of hydroxyapatite. Bone resorption and formation are tightly coupled in each cycle of bone remodeling. Osteoblasts assemble only at sites where osteoclasts have finished the resorption process. (For a detailed discussion of bone remodeling, i.e., bone resorption and formation see Manolagas, Stavros C., 2000, Endocrine Reviews 21, 115-137.)

Cathepsin K is a member of the papain family of cysteine proteases. Cathepsin K is alternatively known as OC2, cathepsin O, cathepsin X, or cathepsin O2. Papain family proteases are expressed in an inactive precursor prepro-form. Cleavage of the amino terminal prepro-leader sequence is necessary for activation of protease activity (Bossard, et. al., 1996 J. Biol. Chem. 271, 12517-12524). Cathepsin S, B, or L are other members of the papain family which were originally suggested as playing a role in osteoclast-mediated resorption. However, it has been shown that cathepsin K is abundantly expressed in osteoclasts, and cathepsins S, B, and L are expressed at very low levels or are absent in osteoclasts (Drake, et. al., 1996, J. Biol. Chem. 271, 12511-12516).

Cathepsin K is unique among the cysteine proteases in that it has the ability to both depolymerize and cleave the insoluble, cross-linked triple helices of type I collagen (Garnero, et. al., 1998, J. Biol. Chem. 273, 32347-32352). The collagenase property of cathepsin K involved in bone resorption is dependent on the formation of a complex of cathepsin K with chondroitin sulfate. Disassociated cathepsin K has no collagenous activity (Li, et. al., 2002 J. Biol. Chem. 277, 28669-28676).

Cathepsin K has been implicated in a number of diseases where the bone resorption/bone formation cycle is imbalanced, including osteoporosis, Paget's disease, and periodontal disease (Rodan, G. A. & Martin, T. J., 2000, Science 289, 1508-1514). As a person ages, bone resorption by osteoclasts outpaces bone formation, resulting in osteoporosis, which is characterized by bone loss and brittle bones that are prone to fracture. Osteoporosis is a major public health problem that affects both menopausal women and older men. Cathepsin K deficiency has also been shown to be the main cause of the autosomal recessive skeletal dysplasia pycnodysostosis (Gelb, et. al., 1996, Science 273, 1236-1238). In addition to bone disorders, cathepsin K has also been associated with the degradation of joint cartilage in rheumatoid arthritis (Hou, et. al., 2001, Am. J. Path. 159, 2167-2177), and with the metastasis of breast cancer tumor cells to bone (Littlewood-Evans, et. al., 1997, Cancer Res. 57, 5386-5390).

Classic inhibitors of cysteine proteases, leupeptin, Z-Phe-Ala-CHN₂, E-64 and cystatin, have been shown to inhibit bone resorption in vitro, while leupeptin and Z-Phe-Ala-CHN₂ have shown some effect in vivo in a murine model of bone resorption (Drake, et. al., 1996, J. Biol. Chem. 271, 12511-12516). Selective inhibitors of cathepsin K have also been developed (Thompson, et. al., 1997, Proc. Natl. Acad. Sci. 94, 14249-14254; U.S. Pat. No. 6,369,077). In addition, cathepsin K antisense oligonucleotides have been shown to reduce bone resorption in vitro (Inui, et. al., 1997, J. Biol. Chem. 272, 8109-8112).

Current therapies for bone disorders involving bone resorption are focused on reducing bone resorption by inhibiting osteoclast activity. Given the major role it plays in bone resorption, inhibition of cathepsin K is recognized as an important therapeutic target (Rodan & Martin, 2000 Science). Inhibition of cathepsin K could prove beneficial in the treatment of osteoporosis, Paget's disease, periodontal disease, pycnodysostosis, rheumatoid arthritis, and breast cancer.

Because of the multiple therapeutic values of drugs targeting cathepsin K (CTSK), there is a need in the art for compounds that selectively bind to isoforms of CTSK. The present invention is directed towards two novel CTSK isoforms (CTSKsv1.1 and CTSKsv1.2) and uses thereof.

### SUMMARY OF THE INVENTION

Microarray experiments and RT-PCR assays have been used to identify and confirm the presence of novel splice variants of human CTSK mRNA. More specifically, the present invention features polynucleotides encoding different protein isoforms of CTSK. A polynucleotide sequence encoding CTSKsv1.1 is provided by SEQ ID NO 1. An amino acid sequence for CTSKsv1.1 is provided by SEQ ID NO 2. A polynucleotide sequence encoding CTSKsv1.2 is provided by SEQ ID NO 3. An amino acid sequence for CTSKsv1.2 is provided by SEQ ID NO 4.

Thus, a first aspect of the present invention describes a purified CTSKsv1.1 encoding nucleic acid and a purified CTSKsv1.2 encoding nucleic acid. The CTSKsv1.1 encoding nucleic acid comprises SEQ ID NO 1 or the complement thereof. The CTSKsv1.2 encoding nucleic acid comprises SEQ ID NO 3 or the complement thereof. Reference to the presence of one region does not indicate that another region is not present. For example, in different embodiments the inventive nucleic acid can comprise, consist, or consist essentially of an encoding nucleic acid sequence of SEQ ID NO 1, or can comprise, consist, or consist essentially of the nucleic acid sequence of SEQ ID NO 3.

Another aspect of the present invention describes a purified CTSKsv1.1 polypeptide that can comprise, consist or consist essentially of the amino acid sequence of SEQ ID NO 2. An additional aspect describes a purified CTSKsv1.2 polypeptide that can comprise, consist, or consist essentially of the amino acid sequence of SEQ ID NO 4.

Another aspect of the present invention describes expression vectors. In one embodiment of the invention, the inventive expression vector comprises a nucleotide sequence encoding a polypeptide comprising, consisting, or consisting essentially of SEQ ID NO 2, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 4, wherein the nucleotide sequence is transcriptionally coupled to an exogenous promoter.

Alternatively, the nucleotide sequence comprises, consists, or consists essentially of SEQ ID NO 1, and is transcriptionally coupled to an exogenous promoter. In another embodiment, the nucleotide sequence comprises, consists, or consists essentially of SEQ ID NO 3, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO 1, SEQ ID NO 3, or a nucleotide sequence encoding a polypeptide comprising, consisting, or consisting essentially of an amino acid sequence of SEQ ID NO 2 or SEQ ID NO 4, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing CTSKsv1.1 or CTSKsv1.2 polypeptide comprising SEQ ID NO 2 or SEQ ID NO 4, respectively. The method involves the step of growing a recombinant cell containing an inventive expression vector under conditions wherein the polypeptide is expressed from the expression vector.

Another aspect of the present invention features a purified antibody preparation comprising an antibody that binds selectively to CTSKsv1.1 as compared to one or more CTSK isoform polypeptides that are not CTSKsv1.1. In another embodiment, a purified antibody preparation is provided comprising antibody that binds selectively to CTSKsv1.2 as compared to one or more CTSK isoform polypeptides that are not CTSKsv1.2.

Another aspect of the present invention provides a method of screening for a compound that binds to CTSKsv1.1, CTSKsv1.2, or fragments thereof. In one embodiment, the method comprises the steps of: (a) expressing a polypeptide comprising the amino acid sequence of SEQ ID NO 2 or a fragment thereof from recombinant nucleic acid; (b) providing to said polypeptide a labeled CTSK ligand that binds to said polypeptide and a test preparation comprising one or more test compounds; (c) and measuring the effect of said test preparation on binding of said test preparation to said polypeptide comprising SEQ ID NO 2. Alternatively, this method could be performed using SEQ ID NO 4 in place of SEQ ID NO 2.

In another embodiment of the method, a compound is identified that binds selectively to CTSKsv1.1 polypeptide as compared to one or more CTSK isoform polypeptides that are not CTSKsv1.1. This method comprises the steps of: providing a CTSKsv1.1 polypeptide comprising SEQ ID NO 2; providing a CTSK isoform polypeptide that is not CTSKsv1.1, contacting said CTSKsv1.1 polypeptide and said CTSK isoform polypeptide that is not CTSKsv1.1 with a test preparation comprising one or more test compounds; and determining the binding of said test preparation to said CTSKsv1.1 polypeptide and to CTSK isoform polypeptide that is not CTSKsv1.1, wherein a test preparation that binds to said CTSKsv1.1 polypeptide but does not bind to said CTSK isoform polypeptide that is not CTSKsv1.1 contains a compound that selectively binds said CTSKsv1.1 polypeptide. Alternatively, the same method can be performed using CTSKsv1.2 polypeptide comprising, consisting, or consisting essentially of SEQ ID NO 4.

In another embodiment of the invention, a method is provided for screening for a compound able to bind to or interact with a CTSKsv1.1 protein or a fragment thereof comprising the steps of: expressing a CTSKsv1.1 polypeptide comprising SEQ ID NO 2 or a fragment thereof from a recombinant nucleic acid; providing to said polypeptide a labeled CTSK ligand that binds to said polypeptide and a test preparation comprising one or more compounds; and measuring the effect of said test preparation on binding of said labeled CTSK ligand to said polypeptide, wherein a test preparation that alters the binding of said labeled CTSK ligand to said polypeptide contains a compound that binds to or interacts with said polypeptide. In an alternative embodiment, the method is performed using CTSKsv1.2 polypeptide comprising, consisting, or consisting essentially of SEQ ID NO 4 or a fragment thereof.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE FIGURES

FIG. 1A illustrates the exon structure of CTSK mRNA corresponding to the known reference form of CTSK mRNA (labeled NM_{—}000396) and the exon structure corresponding to the inventive long form splice variant (labeled CTSKsv1). FIG. 1B depicts the nucleotide sequences of the exon junctions resulting from the splicing of exon 2 to novel exon 2A, and of novel exon 2A to exon 3. In FIG. 1B, in the case of the exon 2-2A junction sequence, the nucleotides shown in italics represent the 20 nucleotides at the 3′ end of exon 2 and the nucleotides shown in underline represent the 20 nucleotides at the 5′ end of exon 2A; in the case of the exon 2A-3 junction sequence, the nucleotides shown in italics represent the 20 nucleotides at the 3′ end of exon 2A and the nucleotides shown in underline represent the 20 nucleotides at the 5′ end of exon 3.

### DEFINITIONS

Unless defined otherwise, all technical and scientific terms used herein have the meaning commonly understood by one of ordinary skill in the art to which this invention belongs.

As used herein, “CTSK” refers to a cysteine protease cathepsin K protein (NP_{—}000387). In contrast, reference to a CTSK isoform, includes NP_{—}000387 and other polypeptide isoform variants of CTSK.

As used herein, “CTSKsv1.1” and “CTSKsv1.2” refer to splice variant isoforms of human CTSK protein, wherein the splice variants have the amino acid sequence set forth in SEQ ID NO 2 (for CTSKsv1.1) and SEQ ID NO 4 (for CTSKsv1.2).

As used herein, “CTSK” refers to polynucleotides encoding CTSK.

As used herein, “CTSKsv1” refers to polynucleotides that are identical to CTSK encoding polynucleotides, except that CTSKsv1 polynucleotides contain additional nucleotides that are not present in CTSK reference messenger RNA NM_{—}000396.2.

As used herein, “CTSKsv1.1” refers to polynucleotides encoding CTSKsv1.1 having an amino acid sequence set forth in SEQ ID NO 2. As used herein, “CTSKsv1.2” refers to polynucleotides encoding CTSKsv1.2 having an amino acid sequence set forth in SEQ ID NO 4.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human CTSKsv1.1 and CTSKsv1.2 that are alternatively spliced isoforms of CTSK, and to the amino acid sequences encoding these proteins. SEQ ID NO 1 and SEQ ID NO 3 are polynucleotide sequences representing exemplary open reading frames that encode the CTSKsv1.1 and CTSKsv1.2 proteins, respectively. SEQ ID NO 2 shows the polypeptide sequence of CTSKsv1.1. SEQ ID NO 4 shows the polypeptide sequence of CTSKsv1.2.

CTSKsv1.1 and CTSKsv1.2 polynucleotide sequences encoding CTSKsv1.1 and CTSKsv1.2 proteins, respectively, as exemplified and enabled herein, include a number of specific, substantial and credible utilities. For example, CTSKsv1.1 and CTSKsv1.2 encoding nucleic acids were identified in an RNA sample obtained from a human source (see Example 1). Such nucleic acids can be used as hybridization probes to distinguish between cells that produce CTSKsv1.1 and CTSKsv1.2 transcripts from human or non-human cells (including bacteria) that do not produce such transcripts. Similarly, antibodies specific for CTSKsv1.1 or CTSKsv1.2 can be used to distinguish between cells that express CTSKsv1.1 or CTSKsv1.2 from human or non-human cells (including bacteria) that do not express CTSKsv1.1 or CTSKsv1.2.

CTSK is a drug target for the treatment of diseases and disorders involving bone resorption such as osteoporosis, Paget's disease, periodontal disease, rheumatoid arthritis and the genetic disorder pycnodysostosis (Rodan, G. A. & Martin, T. J., 2000, Science 289, 1508-1514; Zhenqiang, et. al., 2002, J. Biol. Chem. 277, 28669-28676; Hou, et. al., 2001, Am. J. of Path. 159, 2167-2177; Gelb, et. al., 1996, Science 273, 1236-1238). CTSK has also been found to be expressed in human breast cancer tumor cells and is thought to be involved in breast cancer metastasis to bone tissue (Littlewood-Evans, et. al., 1997, Cancer Res. 57, 5386-5390; Thomas, et. al., 1999, Endocrinology 140, 4451-4458). Given the potential importance of CTSK activity to the therapeutic management of these diseases, it is of value to identify CTSK isoforms and identify CTSK-ligand compounds that are isoform specific, as well as compounds that are effective ligands for two or more different CTSK isoforms. In particular, it may be important to identify compounds that are effective inhibitors of a specific CTSK isoform activity, yet does not bind to or interact with a plurality of different CTSK isoforms. Compounds that bind to or interact with multiple CTSK isoforms may require higher drug doses to saturate multiple CTSK-isoform binding sites and thereby result in a greater likelihood of secondary non-therapeutic side effects. Furthermore, biological effects could also be caused by the interactions of a drug with the CTSKsv1.1 or CTSKsv1.2 isoforms specifically. For the foregoing reasons, CTSKsv1.1 and CTSKsv1.2 proteins represent useful compound binding targets and have utility in the identification of new CTSK-ligands exhibiting a preferred specificity profile and having greater efficacy for their intended use.

In some embodiments, CTSKsv1.1 and CTSKsv1.2 activity is modulated by a ligand compound to achieve one or more of the following: prevent or reduce the risk of occurrence of osteoporosis, Paget's disease, periodontal disease, and rheumatoid arthritis; prevent or reduce the risk of metastasis of breast cancer tumors to bone; or provide treatment for the effects of the genetic disorder pycnodysostosis.

Compounds modulating CTSKsv1.1 or CTSKsv1.2 include agonists, antagonists, and allosteric modulators. While not wishing to be limited to any particular theory of therapeutic efficacy, generally, but not always, CTSKsv1.1 or CTSKsv1.2 compounds may be used to inhibit cysteine protease activity. Inhibitors of CTSK may achieve clinical efficacy by a number of known or unknown mechanisms. In the case of breast cancer metastasis, it is hypothesized that CTSK present in human breast carcinoma cells contributes to degradation of bone extra cellular matrix, thereby facilitating the invasion of osteoclasts by breast tumor cells (Littlewood-Evans, et. al., 1997, Cancer Res. 57, 5386-5390). In the case of rheumatoid arthritis, it has been shown that inhibition of CTSK inhibits cartilage degradation (Hou, et. al., 2001, Am. J. of Path. 159, 2167-2177). In the case of osteoporosis, a disorder characterized by bone loss, it has been suggested that because CTSK is involved in bone resorption, inhibition of CTSK could prevent loss of bone, thereby making CTSK a therapeutic candidate (Rodan, G. A. & Martin, T. J., 2000, Science 289, 1508-1514). Therefore, agents that modulate CTSK activity may be used to achieve a therapeutic benefit for any disease or condition due to, or exacerbated by, abnormal levels of CTSK, or its activity.

CTSKsv1.1 or CTSKsv1.2 activity may also be affected by modulating the cellular abundance of transcripts encoding CTSKsv1.1 or CTSKsv1.2, respectively. Compounds modulating the abundance of transcripts encoding CTSKsv1.1 or CTSKsv1.2 include a cloned polynucleotide encoding CTSKsv1.1 or CTSKsv1.2, respectively, that can express CTSKsv1.2 or CTSKsv1.2 in vivo, antisense nucleic acids targeted to CTSKsv1.1 or CTSKsv1.2 transcripts, and enzymatic nucleic acids, such as ribozymes and RNAi, targeted to CTSKsv1.1 or CTSKsv1.2 transcripts.

In some embodiments, CTSKsv1.1 or CTSKsv1.2 activity is modulated to achieve a therapeutic effect upon diseases in which regulation of cysteine protease activity is desirable. For example, rheumatoid arthritis may be treated by modulating CTSKsv1.1 or CTSKsv1.2 activities to reduce the destruction of joint cartilage. In other embodiments osteoporosis may be treated by modulating CTSKsv1.1 or CTSKsv1.2 activities to inhibit bone resorption. In other embodiments modulation of CTSKsv1.1 or CTSKsv1.2 activities can be used to prevent the metastasis of breast cancer tumors to bone.

CTSKsv1.1 and CTSKsv1.2 Nucleic Acids

CTSKsv1.1 nucleic acids contain regions that encode for polypeptides comprising, consisting, or consisting essentially of SEQ ID NO 2. CTSKsv1.2 nucleic acids contain regions that encode for polypeptides comprising, consisting, or consisting essentially of SEQ ID NO 4. The CTSKsv1.1 and CTSKsv1.2 nucleic acids have a variety of uses, such as use as a hybridization probe or PCR primer to identify the presence of CTSKsv1.1 or CTSKsv1.2 nucleic acids, respectively; use as a hybridization probe or PCR primer to identify nucleic acids encoding for proteins related to CTSKsv1.1 or CTSKsv1.2, respectively; and/or use for recombinant expression of CTSKsv1.1 or CTSKsv1.2 polypeptides, respectively. In particular, CTSKsv1.1 and CTSKsv1.2 polynucleotides have an additional polypeptide encoding region (referred to herein as “exon 2A”) that comprises an alternatively spliced region of intron 2 of the CTSK gene.

Regions in CTSKsv1.1 or CTSKsv1.2 nucleic acid that do not encode for CTSKsv1.1 or CTSKsv1.2, or are not found in SEQ ID NO 1 or SEQ ID NO 3, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding CTSKsv1.1 or CTSKsv1.2 related proteins from different sources. Obtaining nucleic acids encoding CTSKsv1.1 or CTSKsv1.2 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2 Edition, Cold Spring Harbor Laboratory Press, 1989. PCR cloning techniques are described, for example, in White, Methods in Molecular Cloning, volume 67, Humana Press, 1997.

CTSKsv1.1 or CTSKsv1.2 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

CTSKsv1.1 and CTSK1.2 Probes

Probes for CTSKsv1.1 and CTSKsv1.2 contain a region that can specifically hybridize to CTSKsv1.1 or CTSKsv1.2 target nucleic acids, under appropriate hybridization conditions, and can distinguish CTSKsv1.1 or CTSKsv1.2 nucleic acids from non-target nucleic acids, in particular CTSK polynucleotides which lack the additional polynucleotide coding sequence of exon 2A. Probes for CTSKsv1.1 or CTSKsv1.2 may also contain nucleic acid regions that are not complementary with CTSKsv1.1 or CTSKsv1.2 nucleic acids.

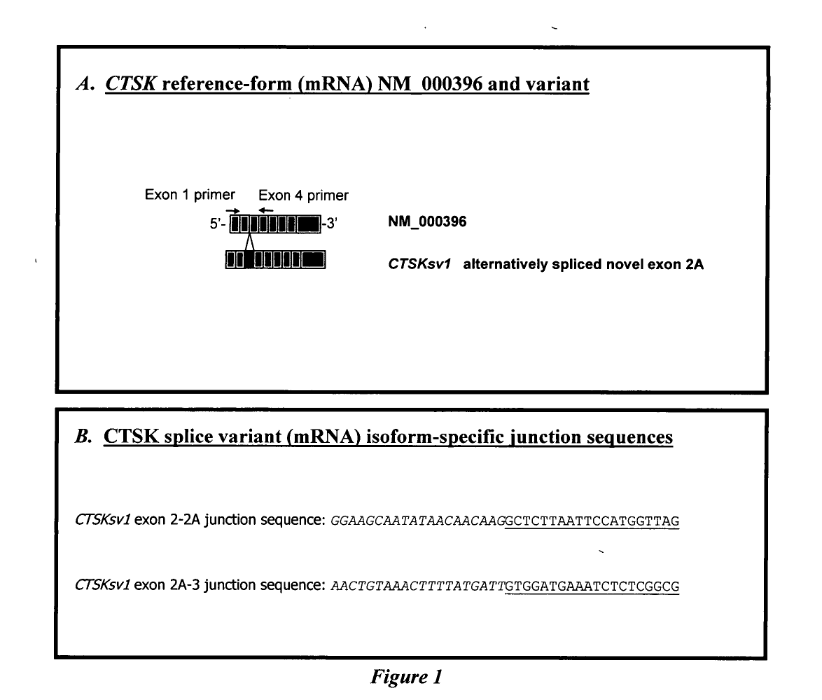

In embodiments where, for example, CTSKsv1.1 and CTSKsv1.2 polynucleotide probes are used in hybridization assays to specifically detect the presence of CTSKsv1.1 or CTSKsv1.2 polynucleotides in samples, the CTSKsv1.1 or CTSKsv1.2 polynucleotides comprise at least 20 nucleotides of the CTSKsv1.1 or CTSKsv1.2 sequence that correspond to the novel exon junction polynucleotide regions. In particular, for detection of CTSKsv1.1, the probe comprises at least 20 nucleotides of the CTSKsv1.1 sequence that corresponds to an exon junction polynucleotide created by the alternative splicing of exon 2 to exon 2A of the CTSK gene (see FIGS. 1A and B). For example, the polynucleotide sequence: 5′ TAACAACAA GGCTCTTAATT 3′ [SEQ ID NO 5] represents one embodiment of such an inventive CTSKsv1.1 polynucleotide wherein a first 10 nucleotide region is complementary and hybridizable to the 3′ end of exon 2 of the CTSK gene and a second 10 nucleotide region is complementary and hybridizable to the 5′ end of alternatively spliced exon 2A of the CTSK gene (see FIG. 1B).

In some embodiments, the first 20 nucleotides of a CTSKsv1.1 probe comprise a first continuous region of 5 to 15 nucleotides that is complementary and hybridizable to the 3′ end of exon 2 and a second continuous region of 5 to 15 nucleotides that is complementary and hybridizable to the 5′ end of exon 2A.

In other embodiments, the CTSKsv1.1 polynucleotide comprises at least 40 or 60 nucleotides of the CTSKsv1.1 sequence that corresponds to a junction polynucleotide region created by the alternative splicing of intron 2 of the CTSK gene, resulting in the splicing of exon 2 to exon 2A. In embodiments involving CTSKsv1.1, the CTSKsv1.1 polynucleotide is selected to comprise a first continuous region of at least 5 to 15 nucleotides that is complementary and hybridizable to the 3′ end of exon 2 and a second continuous region of at least 5 to 15 nucleotides that is complementary and hybridizable to the 5′ end of exon 2A. As will be apparent to a person of skill in the art, a large number of different polynucleotide sequences from the region of the exon 2 to exon 2A splice junction may be selected which will, under appropriate hybridization conditions, have the capacity to detectably hybridize to CTSKsv1.1 polynucleotides, and yet will hybridize to a much less extent or not at all to CTSK isoform polynucleotides lacking exon 2A.

In another embodiment, for detection of CTSKsv1.2, the probe comprises at least 20 nucleotides of the CTSKsv1.2 sequence that corresponds to an exon junction polynucleotide created by the alternative splicing of exon 2A to exon 3 of the CTSK gene (see FIGS. 1A and B). For example, the polynucleotide sequence: 5′ ATGATTGTGGATGAAATCTC 3′ [SEQ ID NO 6] represents one embodiment of such an inventive CTSKsv1.2 polynucleotide wherein a first 6 nucleotide region is complementary and hybridizable to the 3′ end of alternatively spliced exon 2A of the CTSK gene and a second 14 nucleotide region is complementary and hybridizable to the 5′ end of exon 3 of the CTSK gene (see FIG. 1B).

In other embodiments, the CTSKsv1.2 polynucleotide comprises at least 40 or 60 nucleotides of the CTSKsv1.2 sequence that correspond to a junction polynucleotide region created by the alternative splicing of intron 2 of the CTSK gene, resulting in the splicing of exon 2A to exon 3. In embodiments involving CTSKsv1.2, the CTSKsv1.2 polynucleotide is selected to comprise a first continuous region of at least 6 nucleotides that is complementary and hybridizable to the 3′ end of exon 2A and a second continuous region of at least 14 nucleotides that is complementary and hybridizable to the 5′ end of exon 3. As will be apparent to a person of skill in the art, a large number of different polynucleotide sequences from the region of the exon 2A to exon 3 splice junction may be selected which will, under appropriate hybridization conditions, have the capacity to detectably hybridize to CTSKsv1.2 polynucleotides, and yet will hybridize to a much less extent or not at all to CTSK isoform polynucleotides lacking exon 2A.

Preferably, non-complementary nucleic acid that is present has a particular purpose such as being a reporter sequence or being a capture sequence. However, additional nucleic acid need not have a particular purpose as long as the additional nucleic acid does not prevent the CTSKsv1.1 or CTSKsv1.2 nucleic acid from distinguishing between target polynucleotides, e.g. CTSKsv1.1 or CTSKsv1.2 polynucleotides, and non-target polynucleotides, including, but not limited to CTSK polynucleotides lacking exon 2A.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

CTSKsv1.1 or CTSKsv1.2 polynucleotides, such as those comprising SEQ ID NO 1 or SEQ ID NO 3, respectively, can be used to make CTSKsv1.1 or CTSKsv1.2 polypeptides, respectively. In particular, CTSKsv1.1 or CTSKsv1.2 polypeptides can be expressed from recombinant nucleic acids in a suitable host or in vitro using a translation system. Recombinantly expressed CTSKsv1.1 or CTSKsv1.2 polypeptides can be used, for example, in assays to screen for compounds that bind CTSKsv0.1 or CTSKsv1.2, respectively. Alternatively, CTSKsv1.1 or CTSKsv1.2 polypeptides can also be used to screen for compounds that bind to one or more CTSK isoforms but do not bind to CTSKsv1.1 or CTSKsv1.2, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLacl (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen) and pYES2 (Invitrogen), Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) MRC-5 (ATCC CCL 171), and HEK 293 cells (ATCC CRL-1573).

To enhance expression in a particular host it may be useful to modify the sequence provided in SEQ ID NO 1 or SEQ ID NO 3 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

CTSKsv1.1 and CTSKsv1.2 Polypeptides

CTSKsv1.1 polypeptides contain an amino acid sequence comprising, consisting or consisting essentially of SEQ ID NO 2. CTSKsv1.2 polypeptides contain an amino acid sequence comprising, consisting or consisting essentially of SEQ ID NO 4. CTSKsv1.1 or CTSKsv1.2 polypeptides have a variety of uses, such as providing a marker for the presence of CTSKsv1.1 or CTSKsv1.2, respectively; use as an immunogen to produce antibodies binding to CTSKsv1.1 or CTSKsv1.2, respectively; use as a target to identify compounds binding selectively to CTSKsv1.1 or CTSKsv1.2, respectively; or use in an assay to identify compounds that bind to one or more isoforms of CTSK but do not bind to or interact with CTSKsv1.1 or CTSKsv1.2, respectively.

In chimeric polypeptides containing one or more regions from CTSKsv1.1 or CTSKsv1.2 and one or more regions not from CTSKsv1.1 or CTSKsv1.2, respectively, the region(s) not from CTSKsv1.1 or CTSKsv1.2, respectively, can be used, for example, to achieve a particular purpose or to produce a polypeptide that can substitute for CTSKsv1.1 or CTSKsv1.2, or fragments thereof. Particular purposes that can be achieved using chimeric CTSKsv1.1 or CTSKsv1.2 polypeptides include providing a marker for CTSKsv1.1 or CTSKsv1.2 activity, respectively, enhancing an immune response, and modulating cysteine protease activity or levels of CTSK.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional CTSKsv1.1 and CTSKsv1.2

Functional CTSKsv1.1 or CTSKsv1.2 are different protein isoforms of CTSK. The identification of the amino acid and nucleic acid sequences of CTSKsv1.1 or CTSKsv1.2 provide tools for obtaining functional proteins related to CTSKsv1.1 or CTSKsv1.2, respectively, from other sources; for producing CTSKsv1.1 or CTSKsv1.2 chimeric proteins; and for producing functional derivatives of SEQ ID NO 2 or SEQ ID NO 4.

CTSKsv1.1 or CTSKsv1.2 polypeptides can be readily identified and obtained based on their sequence similarity to CTSKsv1.1 (SEQ ID NO 2), or CTSKsv1.2 (SEQ ID NO 4), respectively. In particular, CTSKsv1.1 polypeptides contain 12 consecutive amino acids encoded by alternatively spliced intron 2 sequences, beginning with the nucleotide 322 bases downstream of the 5′ end of intron 2 and ending with the nucleotide 358 bases downstream of the 5′ end of intron 2, of the CTSK gene. The insertion of new coding sequence into the reference CTSK hnRNA transcript (NM_{—}000396.2) results in a peptide region that is unique to the CTSKsv1.1 polypeptide as compared to other known CTSK isoforms. The new coding sequence creates a premature termination codon thirty-six nucleotides downstream of the exon 2/exon 2A splice junction. Thus, CTSKsv1.1 polypeptides are lacking the amino acids encoded by exons 3, 4,5,6,7, and 8 of the CTSK gene.

CTSKsv1.2 polypeptides lack the amino acids encoded by exons 1 and 2 of the CTSK gene, but contain two additional consecutive amino acids encoded by alternatively spliced intron 2 sequence, beginning with the nucleotide 366 bases downstream of the 5′ end of intron 2 and ending with the nucleotide 372 bases downstream of the 5′ end of intron 2, of the CTSK gene. Initiation at a downstream AUG of a bicistronic RNA is a fairly common event and can be associated with disease (Meijer and Thomas, 2002 Biochem. J., 367:1-11; Kozak, 2002, Mammalian Genome 13:401-410).

Both the amino acid and nucleic acid sequences of CTSKsv1.1 or CTSKsv1.2 can be used to help identify and obtain CTSKsv1.1 or CTSKsv1.2 polypeptides, respectively. For example, SEQ ID NO 1 can be used to produce degenerative nucleic acid probes or primers for identifying and cloning nucleic acid polynucleotides encoding for a CTSKsv1.1 polypeptide. In addition, polynucleotides comprising, consisting, or consisting essentially of SEQ ID NO 1 or fragments thereof, can be used under conditions of moderate stringency to identify and clone nucleic acids encoding CTSKsv1.1 polypeptides from a variety of different organisms. The same methods can also be performed with polynucleotides comprising, consisting, or consisting essentially of SEQ ID NO 3, or fragments thereof, to identify and clone nucleic acids encoding CTSKsv1.2.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with CTSKsv1.1 or CTSKsv1.2 obtained from a particular source, derivatives can be produced. Such derivatives include polypeptides with amino acid substitutions, additions and deletions. Changes to CTSKsv1.1 or CTSKsv1.2 to produce a derivative having essentially the same properties should be made in a manner not altering the tertiary structure of CTSKsv1.1 or CTSKsv1.2, respectively.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide than glutamate because of its long aliphatic side chain (See, Ausubel, Current Protocols in Molecular Biology, John Wiley, 1987-1998, Supplement 33 Appendix 1C).

CTSKsv1.1 and CTSKsv1.2 Antibodies

Antibodies recognizing CTSKsv1.1 or CTSKsv1.2 can be produced using a polypeptide containing SEQ ID NO 2 in the case of CTSKsv1.1, or SEQ ID NO 4 in the case of CTSKsv1.2, respectively, or a fragment thereof, as an immunogen. Preferably, a CTSKsv1.1 polypeptide used as an immunogen consists of a polypeptide of SEQ ID NO 2 or a SEQ ID NO 2 fragment having at least 10 contiguous amino acids in length corresponding to the polynucleotide region representing the junction resulting from the alternative splicing of exon 2 to exon 2A of the CTSK gene. Preferably, a CTSKsv1.2 polypeptide used as an immunogen consists of a polypeptide of SEQ ID NO 4 or a SEQ ID NO 4 fragment having at least 10 contiguous amino acids in length corresponding to amino acids, including and downstream of, the amino terminal initiation methionine of CTSKsv1.2.

In other embodiments where, for example, CTSKsv1.1 polypeptides are used to develop antibodies that bind specifically to CTSKsv1.1 and not to other isoforms of CTSK, the CTSKsv1.1 polypeptides comprise at least 10 amino acids of the CTSKsv1.1 polypeptide sequence corresponding to the polynucleotide region representing the junction resulting from the alternative splicing of exon 2 to exon 2A of the CTSK gene. For example, the amino acid sequence: amino terminus-QYNNKALNSM-carboxy terminus [SEQ ID NO 7] represents one embodiment of such an inventive CTSKsv1.1 polypeptide wherein a first 5 amino acid region is encoded by nucleotide sequence at the 3′ end of exon 2 of the CTSK gene and a second 5 amino acid region is encoded by the nucleotide sequence directly after the novel splice junction. Preferably, at least 10 amino acids of the CTSKsv1.1 polypeptide comprises a first continuous region of 2 to 8 amino acids that is encoded by nucleotides at the 3′ end of exon 2 and a second continuous region of 2 to 8 amino acids that is encoded by nucleotides at the 5′ end of exon 2A.

In some embodiments where, for example, CTSKsv1.2 polypeptides are used to develop antibodies that bind specifically to CTSKsv1.2 and not to other isoforms of CTSK, the CTSKsv1.2 polypeptides comprise at least 10 amino acids at the amino terminus of the CTSKsv1.2 polypeptide sequence having at least 10 contiguous amino acids in length corresponding to amino acids, including and downstream of, the amino terminal initiation methionine of CTSKsv1.2. For example, the amino acid sequence: amino terminus-MIVDEISRRL-carboxy terminus [SEQ ID NO 8], represents one embodiment of such an inventive CTSKsv1.2 polypeptide wherein a first 10 amino acid region is encoded by a nucleotide sequence starting with the “AUG” codon 6 nucleotides upstream of the novel exon 2A/exon 3 junction.

In other embodiments, CTSKsv1.1-specific antibodies are made using an CTSKsv1.1 polypeptide that comprises at least 20, 30, 40, or 50 amino acids of the CTSKsv1.1 sequence, wherein twelve amino acids are encoded by a polynucleotide region corresponding to the novel exon 2A coding sequence.

In other embodiments, CTSKsv1.2-specific antibodies are made using a CTSKsv1.2 polypeptide that comprises at least 20, 30, 40 or 50 amino acids of the CTSKsv1.2 sequence that corresponds to a polynucleotide region encoding amino acids, including and downstream of, the initiation methionine codon located six nucleotides upstream of the novel exon 2A/exon 3 splice junction.

Antibodies to CTSKsv1.1 or CTSKsv1.2 have different uses, such as to identify the presence of CTSKsv0.1 or CTSKsv1.2, respectively, and to isolate CTSKsv1.1 or CTSKsv1.2 polypeptides, respectively. Identifying the presence of CTSKsv1.1 can be used, for example, to identify cells producing CTSKsv1.1. Such identification provides an additional source of CTSKsv1.1 and can be used to distinguish cells known to produce CTSKsv1.1 from cells that do not produce CTSKsv1.1. For example, antibodies to CTSKsv1.1 can distinguish human cells expressing CTSKsv1.1 from human cells not expressing CTSKsv1.1 or non-human cells (including bacteria) that do not express CTSKsv1.1. Such CTSKsv1.1 antibodies can also be used to determine the effectiveness of CTSKsv1.1 ligands, using techniques well known in the art, to detect and quantify changes in the protein levels of CTSKsv1.1 in cellular extracts, and in situ immunostaining of cells and tissues. In addition, the same above-described utilities also exist for CTSKsv1.2 specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

CTSKsv1.1 and CTSKsv1.2 Binding Assays

A number of compounds known to inhibit the cysteine protease activity of CTSK have been disclosed (see for example, Delaisse, et al., 1987, Bone 8, 305-313; Lerner, et. al., 1992, J. Bone Miner. Res. 7, 433-439; Thompson, et. al., 1997, Proc. Natl. Acad. Sci. 94, 14249-14254; U.S. Pat. No. 6,369,077). Methods for screening compounds for their effects on the cysteine protease activity of CTSK and on bone resorption have also been disclosed (see for example, Bossard, et. al., 1996, J. Biol. Chem. 21, 12517-12524; U.S. Pat. No. 6,369,077). A person skilled in the art should be able to use these methods to screen CTSKsv1.1 or CTSKsv1.2 polypeptides for compounds that bind to, and in some cases functionally alter, the CTSK isoform protein.

CTSKsv1.1, CTSKsv1.2, or fragments thereof, can be used in binding studies to identify compounds binding to or interacting with CTSKsv1.1, CTSKsv1.2, or fragments thereof, respectively. In one embodiment, the CTSKsv1.1, or a fragment thereof can be used in binding studies with a CTSK isoform protein, or a fragment thereof, to identify compounds that: bind to or interact with CTSKsv1.1 and other CTSK isoforms; or bind to or interact with one or more other CTSK isoforms and not with CTSKsv1.1. A similar series of compound screens can, of course, also be performed using CTSKsv1.2 rather than, or in addition to, CTSKsv1.1. Such binding studies can be performed using different formats including competitive and non-competitive formats. Further competition studies can be carried out using additional compounds determined to bind to CTSKsv1.1, CTSKsv1.2, or other CTSK isoforms.

The particular CTSKsv1.1 or CTSKsv1.2 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using CTSKsv1.1 expressed from a recombinant nucleic acid. Alternatively, recombinantly expressed CTSKsv1.1 consists of the SEQ ID NO 2 amino acid sequence. In addition, binding studies are performed using CTSKsv1.2 expressed from a recombinant nucleic acid. Alternatively, recombinantly expressed CTSKsv1.2 consists of the SEQ ID NO 4 amino acid sequence.

Binding assays can be performed using individual compounds or preparations containing different numbers of compounds. A preparation containing different numbers of compounds having the ability to bind to CTSKsv1.1 or CTSKsv1.2 can be divided into smaller groups of compounds that can be tested to identify the compound(s) binding to CTSKsv1.1 or CTSKsv1.2, respectively.

Binding assays can be performed using recombinantly produced CTSKsv1.1 or CTSKsv1.2 present in different environments. Such environments include, for example, cell extracts and purified cell extracts containing a CTSKsv1.1 or CTSKsv1.2 recombinant nucleic acid; and also include, for example, the use of a purified CTSKsv1.1 or CTSKsv1.2 polypeptide produced by recombinant means which is introduced into different environments.

In one embodiment of the invention, a binding method is provided for screening for a compound able to bind selectively to CTSKsv1.1. The method comprises the steps: providing a CTSKsv1.1 polypeptide comprising SEQ ID NO 2; providing a CTSK isoform polypeptide that is not CTSKsv1.1; contacting the CTSKsv1.1 polypeptide and the CTSK isoform polypeptide that is not CTSKsv1.1 with a test preparation comprising one or more test compounds; and then determining the binding of the test preparation to the CTSKsv1.1 polypeptide and to the CTSK isoform polypeptide that is not CTSKsv1.1, wherein a test preparation that binds to the CTSKsv1.1 polypeptide, but does not bind to CTSK isoform polypeptide that is not CTSKsv1.1, contains one or more compounds that selectively binds to CTSKsv1.1.

In one embodiment of the invention, a binding method is provided for screening for a compound able to bind selectively to CTSKsv1.2. The method comprises the steps: providing a CTSKsv1.2 polypeptide comprising SEQ ID NO 4; providing a CTSK isoform polypeptide that is not CTSKsv1.2; contacting the CTSKsv1.2 polypeptide and the CTSK isoform polypeptide that is not CTSKsv1.2 with a test preparation comprising one or more test compounds; and then determining the binding of the test preparation to the CTSKsv1.2 polypeptide and to the CTSK isoform polypeptide that is not CTSKsv1.2, wherein a test preparation that binds to the CTSKsv1.2 polypeptide, but does not bind to CTSK isoform polypeptide that is not CTSKsv1.2, contains one or more compounds that selectively binds to CTSKsv1.2.

In another embodiment of the invention, a binding method is provided for screening for a compound able to bind selectively to a CTSK isoform polypeptide that is not CTSKsv1.1. The method comprises the steps: providing a CTSKsv1.1 polypeptide comprising SEQ ID NO 2; providing a CTSK isoform polypeptide that is not CTSKsv1.1; contacting the CTSKsv1.1 polypeptide and the CTSK isoform polypeptide that is not CTSKsv1.1 with a test preparation comprising one or more test compounds; and then determining the binding of the test preparation to the CTSKsv1.1 polypeptide and the CTSK isoform polypeptide that is not CTSKsv1.1, wherein a test preparation that binds the CTSK isoform polypeptide that is not CTSKsv1.1, but does not bind the CTSKsv1.1, contains a compound that selectively binds the CTSK isoform polypeptide that is not CTSKsv1.1. Alternatively, the above method can be used to identify compounds that bind selectively to a CTSK isoform polypeptide that is not CTSKsv1.2 by performing the method with CTSKsv1.2 protein comprising SEQ ID NO 4.

The above-described selective binding assays can also be performed with a polypeptide fragment of CTSKsv1.1 or CTSKsv1.2, wherein the polypeptide fragment comprises at least 10 consecutive amino acids that are coded by a nucleotide sequence that bridges the novel junction created by the splicing of the 3′ end of exon 2 to the 5′ end of exon 2A in the case of CTSKsv1.1, or by a nucleotide sequence that bridges the junction created by the splicing of the 3′ end of exon 2A to the 5′ end of exon 3, in the case of CTSKsv1.2. Similarly, the selective binding assays may also be performed using a polypeptide fragment of an CTSK isoform polypeptide that is not CTSKsv1.1, wherein the polypeptide fragment comprises at least 10 consecutive amino acids that are coded by a nucleotide sequence that bridges the junction created by the splicing of the 3′ end of exon 2 to the 5′ end of exon 3 of the CTSK gene.

Cysteine Protease Functional Assays

The identification of CTSKsv1.1 and CTSKsv1.2 as splice variants of CTSK provides a means for screening for compounds that bind to CTSKsv1.1 and/or CTSKsv1.2 protein thereby altering the ability of the CTSKsv1.1 and/or CTSKsv1.2 polypeptide to bind to leupeptin, E-64, cystatin, or any other inhibitor compound, or to perform enzymatic assay for cysteine protease activity, including any CTSK sub-reactions as described, for example in U.S. Pat. Nos. 6,114,132; 6,346,373; 6,348,572; and 6,369,077. Assays involving a functional CTSKsv1.1 or CTSKsv1.2 polypeptide can be employed for different purposes, such as selecting for compounds active at CTSKsv0.1 or CTSKsv1.2; evaluating the ability of a compound to effect cysteine protease activity of each respective splice variant polypeptide; and mapping the activity of different CTSKsv1.1 and CTSKsv1.2 regions. CTSKsv1.1 and CTSKsv1.2 activity can be measured using different techniques such as: detecting a change in the intracellular conformation of CTSKsv1.1 or CTSKsv1.2; detecting a change in the intracellular location of CTSKsv1.1 or CTSKsv1.2; or measuring the level of cysteine protease activity of CTSKsv1.1 or CTSKsv1.2.

Recombinantly expressed CTSKsv1.1 and CTSKsv1.2 can be used to facilitate determining whether a compound is active at CTSKsv1.1 and CTSKsv1.2. For example, CTSKsv1.1 and CTSKsv1.2 can be expressed by an expression vector in a cell line and used in a co-culture growth assay, such as described in WO 99/59037, to identify compounds that bind to CTSKsv1.1 and CTSKsv1.2. For example, CTSKsv1.1 can be expressed by an expression vector in a human kidney cell line 293 and used in a co-culture growth assay, such as described in U.S. Patent Application 20020061860, to identify compounds that bind to CTSKsv1.1. A similar strategy can be used for CTSKsv1.2.

Techniques for measuring cysteine protease activity and substrate specificity are well known in the art. In particular, Bossard, et. al. (1996, J. Biol. Chem. 21, 12517-12524) describe inhibition studies and substrate specificity studies for CTSK; U.S. Pat. Nos. 6,114,132 and 6,348,572 describe use of a scintillation proximity assay (SPA) to determine binding of CTSK; and U.S. Pat. No. 6,346,373 describes a whole cell assay for determining CTSK activity. Other assays can also be used, such as the bone resorption assay described in U.S. Pat. No. 6,369,077.

CTSKsv1.1 or CTSKsv1.2 functional assays can be performed using cells expressing CTSKsv1.1 or CTSKsv1.2 at a high level. These proteins will be contacted with individual compounds or preparations containing different compounds. A preparation containing different compounds where one or more compounds affect CTSKsv1.1 or CTSKsv1.2 in cells over-producing CTSKsv1.1 or CTSKsv1.2 as compared to control cells containing expression vector lacking CTSKsv1.1 or CTSKsv1.2 coding sequences, can be divided into smaller groups of compounds to identify the compound(s) affecting CTSKsv1.1 or CTSKsv1.2 activity, respectively.

CTSKsv1.1 or CTSKsv1.2 functional assays can be performed using recombinantly produced CTSKsv1.1 or CTSKsv1.2 present in different environments. Such environments include, for example, cell extracts and purified cell extracts containing the CTSKsv1.1 or CTSKsv1.2 expressed from recombinant nucleic acid; and the use of a purified CTSKsv1.1 or CTSKsv1.2 produced by recombinant means that is introduced into a different environment suitable for measuring cysteine protease activity.

Modulating CTSKsv1.1 and CTSKsv1.2 Expression

CTSKsv1.1 or CTSKsv1.2 expression can be modulated as a means for increasing or decreasing CTSKsv1.1 or CTSKsv1.2 activity, respectively. Such modulation includes inhibiting the activity of nucleic acids encoding the CTSK isoform target to reduce CTSK isoform protein or polypeptide expressions, or supplying CTSK nucleic acids to increase the level of expression of the CTSK target polypeptide thereby increasing CTSK activity.

Inhibition of CTSKsv1.1 and CTSKsv1.2 Activity

CTSKsv1.1 or CTSKsv1.2 nucleic acid activity can be inhibited using nucleic acids recognizing CTSKsv1.1 or CTSKsv1.2 nucleic acid and affecting the ability of such nucleic acid to be transcribed or translated. Inhibition of CTSKsv1.1 or CTSKsv1.2 nucleic acid activity can be used, for example, in target validation studies.

A preferred target for inhibiting CTSKsv1.1 or CTSKsv1.2 is mRNA stability and translation. The ability of CTSKsv1.1 or CTSKsv1.2 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNAi also can be used to prevent protein expression of a target transcript. This method is based on the interfering properties of double-stranded RNA derived from the coding regions of gene that disrupts the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can effect certain anti-sense activities such as the ability to be cleaved by RNAse H, and can effect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

Increasing CTSKsv1.1 and CTSKsv1.2 Expression

Nucleic acids encoding for CTSKsv1.1 or CTSKsv1.2 can be used, for example, to cause an increase in CTSK activity or to create a test system (e.g., a transgenic animal) for screening for compounds affecting CTSKsv1.1 or CTSKsv1.2 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra. Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of CTSKsv1.1 and CTSKsv1.2 Using Microarrays

To identify variants of the “normal” splicing of the exon regions encoding CTSK, an exon junction microarray, comprising probes complementary to each splice junction resulting from splicing of the 8 exon coding sequences in CTSK heteronuclear RNA (hnRNA), was hybridized to a mixture of labeled nucleic acid samples prepared from 44 different human tissue and cell line samples. Exon junction microarrays are described in PCT patent applications WO 02/18646 and WO 02/16650. Materials and methods for preparing hybridization samples from purified RNA, hybridizing a microarray, detecting hybridization signals, and data analysis are described in van't Veer, et al. (2002 Nature 415:530-536) and Hughes, et al. (2001 Nature Biotechnol. 19:342-7). Inspection of the exon junction microarray hybridization data (not shown) suggested that the structure of at least one of the exon junctions of CTSK mRNA was altered in some of the tissues examined, suggesting the presence of at least one CTSK splice variant mRNA population within the “normal” CTSK mRNA population. Reverse transcription and polymerase chain reactions (RT-PCR) were then performed using oligonucleotide primer sets complementary to exons 1 and exon 4 of the “reference” CTSK mRNA (NM_{—}000396.2) to confirm the exon junction array results and to allow the sequence structure of the splice variants to be determined.

### Example 2

### Confirmation of CTSKsv1.1 and CTSKsv1.2 Using RT-PCR

The structure of CTSK mRNA in the regions spanning exons 1 to 4 was determined for a panel of human tissue and cell line samples using an RT-PCR based assay. PolyA purified mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers were selected that were complementary to sequences in exons 1 and 4 of the reference exon coding sequence in CTSK mRNA (NM_{—}000396.2). Based upon the nucleotide sequence of CTSK mRNA, the CTSK exon 1 and exon 4 primer set (hereafter CTSK₁₋₄ primer set) was expected to amplify a 339 base pairs amplicon representing the “reference” CTSK mRNA region. The CTSK exon 1 forward primer has the sequence: 5′ ACGAAGCCAGACAACAGATTTCCATCAG 3′ [SEQ ID NO: 9]; and the CTSK exon 4 reverse primer has the sequence: 5′ TACTGCGGGAATGAGACAGGGGTA CTTT 3′ [SEQ ID NO: 10].

Twenty-five ng of polyA mRNA from each tissue was subjected to a one-step reverse transcription-PCR amplification protocol using the Qiagen, Inc. (Valencia, Calif.), One-Step RT-PCR kit, using the following conditions:

Cycling conditions were as follows:

50° C. for 30 minutes;

95° C. for 15 minutes;

35 cycles of:

94° C. for 30 seconds;

63.5° C. for 40 seconds;

72° C. for 50 seconds; then

72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

At least two different RT-PCR amplicons were obtained from human mRNA samples using the CTSK₁₋₄ primer set (data not shown). Every human tissue and cell line assayed exhibited the expected amplicon size of 339 base pairs for normally spliced CTSK mRNA. However, in addition to the expected CTSK amplicon of 339 base pairs, all cell lines assayed, except for ileocecum, also exhibited an amplicon of about 390 base pairs. The 390 base pair amplicon was most expressed in cerebellum and cerebral cortex tissue samples. The complete list of tissues in which CTSKsv1 mRNAs were detected is provided in Table 1, wherein an “X” indicates the presence of the about 390 base pair CTSKsv1 amplicon.

TABLE 1

| Sample | CTSKsv1 |
|---|---|
| Heart | x |
| Kidney | x |
| Liver | x |
| Brain | x |
| Placenta | x |
| Lung | x |
| Fetal Brain | x |
| Leukemia Promyelocytic (HL-60) | x |
| Adrenal Gland | x |
| Fetal Liver | x |
| Salivary Gland | x |
| Pancreas | x |
| Skeletal Muscle | x |
| Brain Cerebellum | x |
| Stomach | x |
| Trachea | x |
| Thyroid | x |
| Bone Marrow | x |
| Brain Amygdala | x |
| Brain Caudate Nucleus | x |
| Brain Corpus Callosum | x |
| Heocecum | |
| Lymphoma Burkitt's (Raji) | x |
| Spinal Cord | x |
| Lymph Node | x |
| Fetal Kidney | x |
| Uterus | x |
| Spleen | x |
| Brain Thalamus | x |
| Fetal Lung | x |
| Testis | x |
| Melanoma (G361) | x |
| Lung Carcinoma (A549) | x |
| Adrenal Medula, normal | x |
| Brain, Cerebral Cortex, normal; | x |
| Descending Colon, normal | x |
| Prostate | x |
| Duodenum, normal | x |
| Epididymus, normal | x |
| Brain, Hippocampus, normal | x |
| Ileum, normal | x |
| Interventricular Septum, normal | x |
| Jejunum, normal | x |
| Rectum, normal | x |

Sequence analysis of the about 390 base pair amplicon, herein referred to as “CTSKsv1,” revealed that this amplicon form results from the alternative splicing of intron 2 of the CTSK genomic DNA; that is, CTSKsv1 mRNA contains an additional exon coding sequence in comparison to CTSK reference mRNA NM_{—}000396.2. This novel exon is herein referred to as exon 2A. Thus, the RT-PCR results confirmed the junction probe microarray data reported in Example 1, which suggested that CTSK mRNA is composed of a mixed population of molecules wherein in at least one of the CTSK mRNA splice junctions is altered.

### Example 3

### Cloning of CTSKsv1.1 and CTSKsv1.2

Microarray and RT-PCR data indicate that in addition to the normal CTSK reference mRNA sequence (NM_{—}000396.2), encoding CTSK protein (NP_{—}000387), one novel splice variant form of CTSK mRNA (herein referred to as CTSKsv1) also exists in many tissues.

The polynucleotide sequence of CTSKsv1 mRNA contains two open reading frames that encode an amino terminal and a carboxy terminal protein, referred to herein as CTSKsv1.1 and CTSKsv1.2, respectively. SEQ ID NO 1 encodes the amino terminal CTSKsv1.1 protein (SEQ ID NO 2), similar to the reference CTSK protein (NP_{—}000387), but lacking the amino acids encoded by an 870 base pair region corresponding to exons 3, 4, 5, 6, 7, and 8 of the full length coding sequence of reference CTSK mRNA (NM_{—}000396.2), and including the amino acids encoded by the first 39 base pairs of the novel exon 2A. The alternative spliced CTSKsv1 mRNA not only deletes an 870 base pair region corresponding to exons 3, 4, 5, 6, 7, and 8, but the novel amino acids contain a premature termination codon, resulting in the production of an altered and shorter CTSK protein, referred to herein as CTSKsv1.1, as compared to the reference CTSK protein (NP_{—}000387). In contrast, CTSKsv1.2 polynucleotide (SEQ ID NO 3) encodes a carboxy terminal CTSKsv1.2 protein (SEQ ID NO 4), similar to the reference CTSK protein (NP_{—}000387), but lacking the first 40 amino acids of the reference CTSK protein (NP_{—}000387), and including two amino acids encoded by the last 6 nucleotides of the novel exon 2A. The CTSKsv1.2 protein is produced when a novel translation initiation AUG codon contained in exon 2A and downstream from the reference CTSK protein (NP_{—}000387) AUG initiation codon, is utilized.

A full length CTSK clone having nucleotide sequence comprising the splice variants identified in Example 2 (hereafter referred to as CTSKsv1.1 and CTSKsv1.2) are isolated using a 5′ “forward” CTSK primer and a 3′ “reverse” CTSK primer, to amplify and clone the entire CTSKsv1.1 or CTSKsv1.2 mRNA coding sequences, respectively. The 5′ “forward” CTSKsv1.1 primer is designed for isolation of full length clones corresponding to the CTSKsv1.1 splice variant and has the nucleotide sequence of 5′ ATGTGGGGGCTCAAGGTTCTGCT GCTA 3′ [SEQ ID NO 11]. The 3′ “reverse” CTSKsv1.1 primer is designed to have the nucleotide sequence of 5′ TTACAGTTTAGTTGGGGAACTAACCAT 3′ [SEQ ID NO 12]. The 5′ “forward” CTSKsv1.2 primer is designed for isolation of full length clones corresponding to the CTSKsv1.2 splice variant and has the nucleotide sequence of 5′ ATGATTGTGGATGAA ATCTCTCGGCGT 3′ [SEQ ID NO 13]. The 3′ “reverse” CTSKsv1.2 primer is designed to have the nucleotide sequence of 5′ TCACATCTTGGGGAAGCTGGCCAGGTT 3′ [SEQ ID NO 14].

RT-PCR

The CTSKsv1.1 and CTSKsv1.2 cDNA sequences are cloned using a combination of reverse transcription (RT) and polymerase chain reaction (PCR). More specifically, about 25 ng of human cerebellum polyA mRNA (BD Biosciences Clontech, Palo Alto, Calif.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clontech (Palo Alto, Calif.) Advantage 2 PCR kit. PCR is performed in a Gene Amp PCR System 9700 (Applied Biosystems, Foster City, Calif.) using the CTSK “forward” and “reverse” primers. After an initial 94° C. denaturation of 1 minute, 35 cycles of amplification are performed using a 30 second denaturation at 94° C. followed by a 40 second annealing at 63.5° C. and a 50 second synthesis at 72° C. The 35 cycles of PCR are followed by a 10 minute extension at 72° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determine if the PCR has yielded products of the expected size, in the case of the predicted CTSKsv1.1 and CTSKsv1.2 mRNAs, products of about 159 and 876 bases, respectively. The remainder of the 50 μl PCR reactions from human cerebellum is purified using the QIAquik Gel extraction Kit (Qiagen, Valencia, Calif.) following the QIAquik PCR Purification Protocol provided with the kit. About 50 μl of product obtained from the purification protocol is concentrated to about 6 μl by drying in a Speed Vac Plus (SC 110A, from Savant, Holbrook, N.Y.) attached to a Universal Vacuum Sytem 400 (also from Savant) for about 30 minutes on medium heat.

Cloning of RT-PCR Products

About 4 μl of the 6 μl of purified CTSKsv1.1 and CTSKsv1.2 RT-PCR products from human cerebellum are used in a cloning reaction using the reagents and instructions provided with the TOPO TA cloning kit (Invitrogen, Carlsbad, Calif.). About 2 μl of the cloning reaction is used following the manufacturer's instructions to transform TOP 10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2nd Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit. Twelve putative CTSKsv1.1 and CTSKsv1.2 clones, respectively, are identified and prepared for a PCR reaction to confirm the presence of the expected novel CTSKsv1.1 and CTSKsv1.2 exon 2A coding sequence. A 25 μl PCR reaction is performed as described above (RT-PCR section) to detect the presence of CTSKsv1.1, except that the reaction includes miniprep DNA from the TOPO TA/CTSKsv1.1 ligation as a template. An additional 25 μl PCR reaction is performed as described above (RT-PCR section) to detect the presence of CTSKsv1.2, except that the reaction includes miniprep DNA from the TOPO TA/CTSKsv1.2 ligation as a template. About 10 μl of each 25 μl PCR reaction is run on a 1% Agarose gel and the DNA bands generated by the PCR reaction are visualized and photographed on a UV light box to determine which minipreps samples have PCR product of the size predicted for the corresponding CTSKsv1.1 and CTSKsv1.2 splice variant mRNAs. Clones having the CTSKsv1.1 structure are identified based upon amplification of an amplicon band of 159 basepairs. Clones having the CTSKsv1.2 structure are identified based upon amplification of an amplicon band of 876 basepairs. DNA sequence analysis of the CTSKsv1.1 cloned DNAs confirm a polynucleotide sequence representing the absence of exons 3, 4, 5, 6, 7, and 8, plus the addition of 39 nucleotides of novel exon 2A sequence. DNA sequence analysis of the CTSKsv1.2 cloned DNAs confirm a polynucleotide sequence representing the absence of exons 1 and 2, plus the addition of 6 nucleotides of novel exon 2A sequence.

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed:

1. A purified human nucleic acid comprising SEQ ID NO 3, or the complement thereof.

2. The purified nucleic acid of claim 2, wherein said nucleic acid comprises a region encoding SEQ ID NO 4.

3. The purified nucleic acid of claim 2, wherein said nucleotide sequence encodes a polypeptide consisting of SEQ ID NO 4.

4. A purified polypeptide comprising SEQ ID NO 4.

5. The polypeptide of claim 4, wherein said polypeptide consists of SEQ ID NO 4.

6. An expression vector comprising a nucleotide sequence encoding SEQ ID NO 4, wherein said nucleotide sequence is transcriptionally coupled to an exogenous promoter.

7. The expression vector of claim 6, wherein said nucleotide sequence encodes a polypeptide consisting of SEQ ID NO 4.

8. The expression vector of claim 6, wherein said nucleotide sequence comprises SEQ ID NO 3.

9. The expression vector of claim 6, wherein said nucleotide sequence consists of SEQ ID NO 3.

10. A method for screening for a compound able to bind to CTSKsv1.2 comprising the steps of:
(a) expressing a polypeptide comprising SEQ ID NO 4 from recombinant nucleic acid;
(b) providing to said polypeptide a test preparation comprising one or more test compounds; and
(c) measuring the ability of said test preparation to bind to said polypeptide.

11. The method of claim 10, wherein said steps (b) and (c) are performed in vitro.

12. The method of claim 10, wherein said steps (a), (b), and (c) are performed using a whole cell.

13. The method of claim 10, wherein said polypeptide is expressed from an expression vector.

14. The method of claim 10, wherein said polypeptide consists of SEQ ID NO 4.

15. A method of screening for compounds able to bind selectively to CTSKsv1.2 comprising the steps of:
(a) providing a CTSKsv1.2 polypeptide comprising SEQ ID NO 4;
(b) providing one or more CTSK isoform polypeptides that are not CTSKsv1.2;
(c) contacting said CTSKsv1.2 polypeptide and said CTSK isoform polypeptide that is not CTSKsv1.2 with a test preparation comprising one or more compounds; and
(d) determining the binding of said test preparation to said CTSKsv1.2 polypeptide and to said CTSK isoform polypeptide that is not CTSKsv1.2, wherein a test preparation which binds to said CTSKsv1.2 polypeptide, but does not bind to said CTSK polypeptide that is not CTSKsv1.2, contains a compound that selectively binds said CTSKsv1.2 polypeptide.

16. The method of claim 15, wherein said CTSKsv1.2 polypeptide is obtained by expression of said polypeptide from an expression vector comprising a polynucleotide encoding SEQ ID NO 4.

17. The method of claim 15, wherein said polypeptide consists of SEQ ID NO 4.

18. A method for screening for a compound able to bind to or interact with a CTSKsv1.2 protein or a fragment thereof comprising the steps of:
(a) expressing a CTSKsv1.2 polypeptide comprising SEQ ID NO 4 or fragment thereof from a recombinant nucleic acid;
(b) providing to said polypeptide a labeled CTSK ligand that binds to said polypeptide and a test preparation comprising one or more compounds; and
(c) measuring the effect of said test preparation on binding of said labeled CTSK ligand to said polypeptide, wherein a test preparation that alters the binding of said labeled CTSK ligand to said polypeptide contains a compound that binds to or interacts with said polypeptide.

19. The method of claim 18, wherein said steps (b) and (c) are performed in vitro.

20. The method of claim 18, wherein said steps (a), (b) and (c) are performed using a whole cell

21. The method of claim 18, wherein said polypeptide is expressed from an expression vector

22. The method of claim 21, wherein said expression vector comprises SEQ ID NO 3 or a fragment of SEQ ID NO 3.

23. The method of claim 21, wherein said polypeptide comprises SEQ ID NO 4 or a fragment of SEQ ID NO 4.
